(19) United States
(12) Patent Application Publication
(10) Pub. No.: US 2014/0216350 A1
(43) Pub. Date: Aug. 7, 2014

(54) ARTIFICIAL PAIRING AND BREEDING METHOD OF PLATYSTERNON MEGACEPHALUM

(71) Applicant: Guangdong Entomological Institute, Guangzhou City (CN)

(72) Inventors: Shiping Gong, Guangzhou City (CN); Yan Ge, Guangzhou City (CN); Xiangjing Zhong, Guangzhou City (CN)

(73) Assignee: Guangdong Entomological Institute, Guangzhou City (CN)

(21) Appl. No.: 14/245,183

(22) Filed: Apr. 4, 2014

Related U.S. Application Data
(63) Continuation of application No. PCT/CN2013/076624, filed on Jun. 3, 2013.

(30) Foreign Application Priority Data
Jul. 13, 2012 (CN) 201210244404.6

(51) Int. Cl. A01K 67/02

(52) U.S. Cl. CPC A01K 67/02
USPC 119/200

(57) ABSTRACT

The invention discloses an artificial pairing and breeding method of Platysternon megacephalum. The method comprises the selection of species sources, domestication before pairing, setting of a breeding pond, artificial pairing, collection and hatching of turtle eggs. The method fully considers the ecological and biological characteristics of the Platysternon megacephalum, including the behavior, feeding habit, habitat, and breeding ecology. The main problems in artificial pairing and captive breeding of the Platysternon megacephalum can be effectively solved, including the breeding turtle selection, pairing, pairing stage selection, sex ratio, density, artificial food, setting of the reproduction and hatching environments, the occurrence of disabilities and fatal events during the pairing and mating process is significantly reduced; the egg laying rate of female turtles and the hatching rate of turtle eggs are both significantly improved. Artificial high-efficiency breeding of the Playsternon megacephalum is realized, and healthy turtle hatchlings can be obtained.

### FIELD OF THE INVENTION

The invention belongs to the field of artificial domestication and breeding of wild animals and specifically relates to an artificial pairing and breeding method of Platysternon megacephalum.

### BACKGROUND OF THE INVENTION

Platysternon megacephalum is a rare economic freshwater turtle which is an indigenous species in Asia. The Platysternon megacephalum is distributed in southeastern Asian, China (various provinces in South China, East China and Southwest China, as well as Hong Kong), Thailand, Vietnam, Cambodia, Laos, and Myanmar. They prefer to choose habitats in rocky mountain streams with thick vegetation in the altitude range of 200-1700 meters. Compared with other freshwater turtles, the Platysternon megacephalum likes cool water and has ferocious and aggressive temper and stronger territorial behaviors.

The Platysternon megacephalum has great economic value as food and medicine, and has been over-hunted and traded for a long time, decreasing the wild population drastically, and driving the Platysternon megacephalum to be endangered. Domestication and captive breeding of the Platysternon megacephalum can not only effectively increase the population number and avoid this species extinction, but also can protect the wild resources and realize sustained utilization of the valuable resources. As an excellent new freshwater turtle breeding species, the captive breeding of the Platysternon megacephalum draws more and more attention in the aquaculture industry However, as the Platysternon megacephalum has great differences in their requirements on water quality, water temperature, food, breeding density, sex ratio, environmental conditions etc. relative to other freshwater turtles, the successful mating and breeding of the species in captivity is very difficult, seriously restricting the development of the breeding industry of Platysternon megacephalum.

At present, the main problems in the artificial pairing and breeding of the Platysternon megacephalum include: (1) the age of sex maturity of the Platysternon megacephalum is undefined, bring problems in the selection and pairing of the breeding turtles; (2) the optimal mating period is not clear; (3) the matching, the sex ratio and the density setting of the female and male breeding individuals are unreasonable, thereby resulting in mutual disabilities, diseases and deaths; and (4) the setting of the breeding environments (hiding places, egg laying places and activity spaces) is unreasonable, the water quality and water temperature are improper, and the feeding is unreasonable, resulting in behavioral and physiological abnormalities, diseases and incapability of mating and breeding.

### SUMMARY OF THE INVENTION

The invention aims to provide an artificial pairing and breeding method of Platysternon megacephalum. This method is effective in solving a series of technical problems in captive breeding of the Platysternon megacephalum, including artificial pairing and breeding. Healthy turtle hatchlings are obtained through this method, which could provide great technical supports for the large-scale captive breeding of Platysternon megacephalum.

This invention provides a set of artificial pairing and breeding method of the Platysternon megacephalum based on years of experimental studies and fully consideration of the ecological and biological characteristics of the Platysternon megacephalum, including the behavior, feeding habit, habitat, breeding ecology and the like. By applying this method, the main problems in artificial pairing and captive breeding of the Platysternon megacephalum can be effectively solved, including the breeding turtle selection, pairing, pairing stage selection, sex ratio, density, artificial food, setting of the reproduction and hatching environments and the like, the occurrence of disabilities and fatal events during the pairing and mating process is significantly reduced (above 95% of the individuals are safe during the breeding period); the egg laying rate of female turtles and the hatching rate of turtle eggs are both significantly improved, more than 80% of the female turtles have oviposition behaviors, and more than 85% of the turtles eggs hatched successfully each year. Generally, by adopting this method, the artificial high-efficiency breeding of the Platysternon megacephalum is realized, and healthy turtle hatchlings can be obtained. This method may provide technical supports for expanding breeding populations, promoting species conservation and developing large-scale breeding of the Platysternon megacephalum. Additionally, this method has the advantages of low human investment, low cost and easiness in operation, and is conductive to popularization of captive breeding of the Platysternon megacephalum.

### BRIEF DESCRIPTION OF THE DRAWINGS

FIG. 1 is a schematic diagram of the structure of a breeding pond of embodiment 1.

### DETAILED DESCRIPTION OF THE INVENTION

The following embodiment is used for further describing the invention rather than limiting the invention.

### Embodiment 1

The artificial pairing and breeding method of Platysternon megacephalum is characterized by comprising the following steps (a)-(e).

(a) Selection of species sources.

Breeding turtles of the Platysternon megacephalum are caught from the field by adopting a harmless trapping method. The candidates should be sexually mature and have the ability to reproduce. They also should have robust health with sensitive actions, bright eyes, no diseases and disabilities, no deformities, intact and defect-free tails and four limbs and no damages or ulcers in skins, carapace and plastron. The body weight of female turtles is required to be above 300 g (300-500 g in this embodiment), and the body weight of male turtles is required to be above 400 g (400-500 g in this embodiment). The sex identification of these breeding turtles is based on the positions of their cloacal apertures, when their tails are stretched outwards along the midlines of the plastron, the cloacal apertures of the female turtles do not exceed the back edges of their carapaces, while the cloacal apertures of the male turtles exceed the back edges of their carapaces.

(b) Domestication before pairing.

The breeding turtles of Platysternon megacephalum have strong wildness, show obvious stress response at the beginning of domestication and generally have anti-feeding effect within the initial 7-15 days. To domesticate the turtles, they are first raised in a water pool of 1-2 m², providing a quiet environment and clean water quality with the pH value at 5-7, the water temperature at 20-26° C. and the water depth at 15-30 cm. Aquatic plants are planted and stone caves are created in the water pool for sheltering the turtles. Each turtle has its own individual cave, and all the turtles and their caves are domesticated in one pool. That is, there are some caves in the pool, and some aquatic plants are planted among these caves, so that the aquatic plants can prevent the turtles from communicating with each other. A stone platform is provided with the top which is 3-10 cm higher than the water level and an inclined plane for enabling the turtles to climb up for airing the bodies. The turtles are fed with fresh loathes, eels and mountain stream fishes since they start eating, and accompanied with river snails, river shrimps and animal livers; enabling the turtles to hibernate naturally in winter. The domestication is performed for more than 1 year, thereby adapting these turtles to the captive breeding environment. The turtles are then used for pairing and breeding.

(c) Setting of a breeding pond.

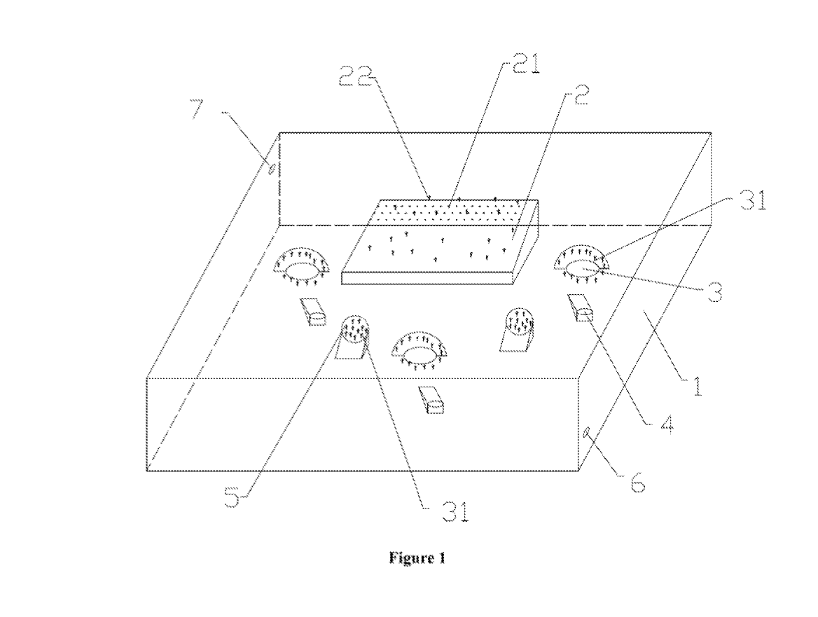

The breeding pond is constructed in a mountain area with appropriate climate, wherein, the area of the breeding pond is preferably 6-18 m² (6 m² and rectangular in this embodiment). The breeding pond is illustrated in FIG. 1, where 1 is a water pool, 2 is an egg laying area, 21 is silt, 22 is a plant grown in a moisture environment, 3 is a hidden cave, 31 is an aquatic plant, 4 is a feeding platform, 5 is a green island, 6 is a water outlet, and 7 is a water inlet. An “aquatic plant” is a plant which grows in water. A “plant grown in a moisture environment” is a plant that can brow in a humid environment (a hygrophyte). The breeding pond includes a water pool 1, wherein the water depth is 20-50 cm, the wall of the water pool is smooth and the top is 30-50 cm higher than the water level, a water inlet is placed at the upper side of the wall, and a water outlet is placed at the bottom of the other opposite side of the wall, the water quality is clean with the pH value at 5-7 and the water temperature at 20-26° C. Three hidden caves 3 and an egg laying area 2 was set in the water pool, wherein the egg laying area 2 is positioned at the middle part of the water pool 1 near the upper side. “Hidden caves” means a cave which is suitable for a turtle to hide in. The egg laying area 2 is 5-10 cm higher than the water level, and an inclined plane is set at the part which is contiguous to the water level. Fine and soft silt 21 is spread on the egg laying area and accumulated to form a small approximately rectangular or circular land with an area of 1-2 in², with edges building with bricks for preventing the silt from flowing into the water. The water can wet the silt. Plants 22 grown in a moisture environment, such as Acorus calamus, Herba houttuyniae, pteridophytes and the like are planted on the egg laying area 2, the plants intervals are kept at 15-20 cm, providing good places for enabling the turtles to lay eggs.

The hidden caves 3 are dispersedly arranged and constituted by relatively smooth stone blocks or bricks, the depth of the hidden caves 3 is 2-3 times the length of turtle's carapace, the width of 2 times of the turtle's carapace width, and the height of 2 times of the turtle's body height. The tops of the hidden caves 3 are 5-10 cm lower than the water level, with aquatic plants 31 (such as Acorus calamus, Acorns gramineus and the like) planted at the tops and the peripheries. Green islands 5 are arranged between two hidden caves, and the aquatic plants 31, such as the Acorns calamus and the Acorus gramineus are planted on the green islands 5 for reinforcing the hiding effect. A feeding platform 4 is set in front of a cave opening within the range of 20-50 cm. The feeding platforms 4 are 1 cm higher than the water level and have an area of about 20 cm×20 cm. The inclined plane is placed on the side of each feeding platform 4, close to the hidden cave, for enabling the turtles to climb up the feeding platform for intake, and the feeding platform can also be simultaneously used for the turtles to air their bodies. A water inlet 7 is set at the upper side of the wall of the water pool, and a water outlet 6 is set at the bottom of the other opposite side of the wall, so as to enable the mountain spring water to enter from the water inlet 7 and exit from the water outlet 6. The water inflow and the water outflow are kept equivalent, and the daily water inflow is preferably 1-2 times the water volume of the turtle pond.

(d) Artificial pairing.

Pairing of the turtles is started after the end of hibernation of the Platysternon megacephalum when the temperature rises to above 20° C. The well domesticated Platysternon megacephalum are placed in the breeding pond, with a male-female proportion of 1:2-3. The body weight and the carapace length the male turtles are required to be larger than those of the female turtles, the density of the turtles cannot be higher than 0.5 turtle/m², and the number of the hidden caves is no less than that of the turtles. The breeding turtles are allowed to perform mutual contacts, competitions and domain distributions within 10-20 days after pairing. During this period, turtles with serious fighting and biting are replaced by the turtles with the same size, until the breeding turtles can coexist safely. The turtles are fed on the feeding platforms, with the fresh loathes as the main foods, and accompanied by the river snails, crabs, the river shrimps and the animal (swine and bovine) livers. Fixed-time and fixed-location feeding is performed, which is feeding once at 6:00-7:00 at night every 2-3 days. The food quantity is subject to the situation that there are little remains after the feeding of the turtles, and the remaining food is cleaned in time to avoid decaying and polluting the water quality. The mating activities are completed within 2-3 months after pairing.

(e) Collection and hatching of turtle eggs.

The Platysternon megacephalum is used to laying eggs in May-September. The females dig nests for laying eggs in the silt below grass in the egg laying area, wherein the depth of the nests is about 5 cm. Eggs laid in water by the turtles are timely picked out. The eggs are gently collected and placed in an incubator to hatch. Fine sand or vermiculite after high-temperature disinfection is used as a hatching medium. The eggs are flatly placed on the surface of the hatching medium with a thickness of 5-10 cm, then covered with hatching medium of 1-2 cm. The relative humidity of the medium is kept at 70-80%, and the hatching temperature is kept at 25-27° C. until the turtles hatch. The hatching period is about 60 days.

According to the embodiment, no obvious disabilities and fatal events occur during the pairing and mating process, 6 female turtles lay eggs, and the hatching rate of the turtle eggs is 87%. Generally, by adopting this method, the artificial high-efficiency breeding of the Platysternon megacephalum is realized, and healthy turtle hatchlings can be obtained. This method may provide technical support for expanding breeding populations, promoting species conservation and developing large-scale breeding of the Platysternon megacephalum. Additionally, this method has the advantages of low human investment, low cost and ease of operation, and is conductive to popularization of captive breeding of the Platysternon megacephalum.

Although a specific form of embodiment of the instant invention has been described above and illustrated in the accompanying drawings in order to be more clearly understood, the above description is made by way of example and not as a limitation to the scope of the instant invention. It is contemplated that various modifications apparent to one of ordinary skill in the art could be made without departing from the scope of the invention which is to be determined by the following claims.

We claim:

1. An artificial pairing and breeding method of Platysternon megacephalum, comprising:
(a) obtaining Platysternon megacephalum turtles, the turtles being sexually mature,
(b) domesticating the turtles in a domestication environment,
(c) providing a breeding pond including a plurality of egg laying areas and hidden caves,
(d) artificial pairing the turtles after a hibernation period by placing the domesticated turtles in the breeding pond, when the temperature is 20° C. or higher, and
(e) collecting turtle eggs laid in the egg laying areas, and placing the turtle eggs in an incubator.

2. The artificial pairing and breeding method according to claim 1,
wherein the body weight of female turtles in step (a) is 300 grams or more, and
wherein the body weight of male turtles in step (a) is 400 grams or more.

3. The artificial pairing and breeding method according to claim 1,
wherein the domestication environment comprises
a clean water pool having an area of 1-2 m², a pH value of 5-7, a water temperature of 20-26° C., and a water depth at 15-30 cm,
aquatic plants or plants grown in a moisture environment,
stone caves,
a stone platform having a top which is 3-10 cm higher than the water level, and
an inclined plane.

4. The artificial pairing and breeding method according to claim 1,
wherein, in step (b), the turtles are fed a diet comprising one or more of fresh loaches, eels, mountain stream fish, and river snails, river shrimps and animal livers, and
wherein, the domestication is performed for at least 1 year.

5. The artificial pairing and breeding method according to claim 1,
wherein the breeding pond comprises
a clean water pool having a depth of 20-50 cm,
a smooth wall with a top thereof being 30-50 cm higher than the water level,
a water inlet disposed in an upper part of the wall, and
a water outlet disposed at a bottom part of the wall, at an opposite side from the water inlet, and
wherein the water in the clean water pool of the breeding pond has a pH value at 5-7 and a water temperature of 20-26° C.

6. The artificial pairing and breeding method according to claim 1,
wherein, in the breeding pond, the egg laying areas comprise
silt,
aquatic plants or plants grown in a moisture environment at an interval of 15-20 cm, and
an inclined platform, and
wherein, in the breeding pond, the egg laying areas are 5-10 cm higher than the water level.

7. The artificial pairing and breeding method according to claim 1,
wherein, in the breeding pond, the hidden caves have a depth of 2-3 times a carapace length of the turtles, a width of 2 times a carapace width of the turtle, and a height of 2 times a body height of the turtles, wherein, in the breeding pond, the hidden caves have tops thereof which are 5-10 cm lower than the water level, and
wherein, in the breeding pond, the hidden caves have aquatic plants or plants grown in a moisture environment planted at the tops and peripheries of thereof.

8. The artificial pairing and breeding method according to claim 1,
wherein the breeding pond further comprises feeding platforms disposed within 20-50 cm in front of each hidden cave, and
wherein the feeding platforms are 1 cm higher than the water level and include an inclined platform.

9. The artificial pairing and breeding method according to claim 1,
wherein, in step (d), the turtles in the breeding pond have a male-female proportion of 1:2 to 1:3,
wherein, in step (d), the density of the turtles is not higher than 0.5 turtle/m², and
wherein, in step (d), the number of turtles is no less than the number of hidden caves in the breeding pond.

10. The artificial pairing and breeding method according to claim 1,
wherein, in step (d), during 10-20 days after pairing, turtles with fighting and biting behavior are replaced with turtles having the same size.

11. The artificial pairing and breeding method according to claim 1,
wherein, in step (d), the turtles are fed on the feeding platforms with a diet comprising at least one of fresh loaches, river snails, crabs, river shrimps and animal livers,
wherein, in step (d), feeding is at a fixed time and fixed location every 2-3 days, and
wherein, in step (d),food remaining after feeding is removed.

12. The artificial pairing and breeding method according to claim 1,
wherein, in step (e), fine sand or vermiculite which has been subjected to high-temperature disinfection is used as a hatching medium,
wherein, in step (e), in the incubator, the turtle eggs are placed on the surface of a first layer of the hatching medium which is 5-10 cm thick, and the turtle eggs are covered by a second layer of the hatching medium which is 1-2 cm thick, and
wherein, in step (e), the relative humidity in the incubator is 70-80%, and the temperature in the incubator is 25-27° C.

13. The artificial pairing and breeding method according to claim 3,
wherein the aquatic plants are Acorus calamus and/or Acorns gramineus, and
wherein the plants grown in the moisture environment are Acorns gramineus, Herba houttuyniae and/or pteridophytes.

14. The artificial pairing and breeding method according to claim 6,
wherein the aquatic plants are Acorus calamus and/or Acorus gramineus, and wherein the plants grown in the moisture environment are Acorus gramineus, Herba houttuyniae and/or pteridophytes.

15. The artificial pairing and breeding method according to claim 7,
wherein the aquatic plants are Acorus calamus and/or Acorus gramineus, and
wherein the plants grown in the moisture environment are Acorus gramineus, Herba houttuyniae and/or pteridophytes.

16. The artificial pairing and breeding method according to claim 1, wherein the plurality of egg laying areas are disposed on one side of the breeding pond, or at a middle part of the breeding pond.

17. The artificial pairing and breeding method according to claim 1, wherein the area of the breeding pond is 6-18 m², and water in the breeding pond is mountain spring water.

18. The artificial pairing and breeding method according to claim 4, wherein the mountain stream fish include Misgurnus anguillicaudatus and Monopterus albus.
